(19) United States
(12) Patent Application Publication
(10) Pub. No.: US 2025/0198975 A1
(43) Pub. Date: Jun. 19, 2025

(54) SYSTEM AND METHOD FOR GAS SENSOR DETERMINATION OF VOLATILE COMPOUNDS BY METERED SAMPLE EVAPORATION

(71) Applicant: RARE COMBINATIONS LLC, Riverside, CA (US)

(72) Inventor: Nicholas R. Robertson, Riverside, CA (US)

(21) Appl. No.: 18/616,779

(22) Filed: Mar. 26, 2024

Related U.S. Application Data
(60) Provisional application No. 63/612,347, filed on Dec. 19, 2023.

(51) Int. Cl. G01N 33/00

(52) U.S. Cl. CPC G01N 33/0011; G01N 33/0006; G01N 33/0062; G01N 33/0068

(57) ABSTRACT

A detection system for measuring the concentration of a volatile or gaseous compound in a liquid comprising a reservoir configured to receive the liquid; an evaporator configured to evaporate or vaporize the liquid received in the reservoir into a gas including an amount of a liquid sample; one or more gas sensors coupled to the evaporator and configured to sense the amount of the liquid sample in the gas to measure the concentration of the volatile or gaseous compound.

### CROSS-REFERENCE TO RELATED APPLICATION

This application claims the benefit of U.S. Provisional Patent Application No. 63/612,347, filed Dec. 19, 2023, under 35 U.S.C. 119, which is incorporated by reference herein.

### FIELD OF THE INVENTION

The present disclosure generally relates to alcohol testing devices and analytical chromatography, and methods.

### BACKGROUND OF THE INVENTION

Compact, easy to use, and affordable testing methods can expand quality control capabilities in multiple industries. For example, kombucha brewers typically create a naturally fermented product that contains relatively low levels of alcohol that often exceed regulatory requirements for non-alcoholic products. Due to the high levels of acidity and low levels of alcohol, traditional beer or wine brewing testing methods simply do not provide accurate results for kombucha. The only viable option for ensuring low alcohol levels is laboratory testing that uses bulky and expensive gas chromatography. There exists a need for rapid, in-house alcohol testing for kombucha brewers.

Similar problems also exist in the production of all other beverages. Fruit juices contain low levels of alcohol, beer may be barrel aged which obscures more affordable alcohol measurements, spirits often have added sugars or flavors that interfere with common high-alcohol measuring equipment, and most wine producers rely on slow ebulliometer testing methods. The beverage industry has a need for an affordable, rapid, and compact alcohol testing device.

### SUMMARY OF THE INVENTION

An aspect of the present disclosure involves a device for measuring the concentration of a volatile or gaseous compound(s) in a liquid. The device operates by evaporating or vaporizing a metered amount of a liquid sample which is carried by a gas. The compound-enriched gas is then directed over a gas sensor or sensor array. The gas sensor(s) provides a varying voltage output proportional to the compound near the sensor. A microcontroller controls system elements and collects raw sensor values. Through calculations, the exact amount of compound in the original sample is determined, based on the integral of the sensor readings. This measurement remains negligibly unaffected by atmospheric temperature, pressure, sample temperature, sample viscosity, or off-target compounds in the sample. The device presents the results on a display or a connected application, offering clear information to the user. This disclosure provides a simple alternative to costly and bulky lab techniques like gas chromatography.

Another aspect of the present disclosure involves a detection system for measuring the concentration of a volatile or gaseous compound in a liquid comprising a reservoir configured to receive the liquid; an evaporator configured to evaporate or vaporize the liquid received in the reservoir into a gas including an amount of a liquid sample; one or more gas sensors coupled to the evaporator and configured to sense the amount of the liquid sample in the gas to measure the concentration of the volatile or gaseous compound.

One or more implementations of the aspect of the present disclosure described immediately above includes one or more of the following: the one or more sensors are configured to provide a varying voltage output proportional to a compound in the amount of the liquid sample in the gas near the one or more sensors, the detection system further including a controller configured to receive sensor values from the one or more gas sensors and calculate an amount of compound in the liquid based on an integral of the sensor values received from the one or more gas sensors; the detection system is configured to measure the concentration of the volatile or gaseous compound in the liquid independent of atmospheric temperature, atmospheric pressure, sample temperature, sample viscosity, off-target compounds, or other environmental variables in the liquid; the detection system is configured to measure the concentration of the volatile or gaseous compound in the liquid negligibly unaffected by viscosity, carbonation, turbidity, sugar content, or acid content of the liquid; the detection system is configured to meter one or more of the liquid and the gas via one or more of volumetric measurement of the liquid, limiting evaporation or vaporization time, volumetric airflow measurement, and mass measurement; the evaporator includes an ultrasonic disk configured to vaporize the liquid; and/or the ultrasonic disk is configured to vaporize the liquid into one of faster vaporization yielding a jet of vapor mist and slower, pulsed vaporization.

An additional aspect of the present disclosure involves a method of measuring the concentration of a volatile or gaseous compound in a liquid comprising providing a detection system for measuring the concentration of a volatile or gaseous compound in a liquid comprising a reservoir configured to receive the liquid; an evaporator configured to evaporate or vaporize the liquid received in the reservoir into a gas including an amount of a liquid sample; one or more gas sensors coupled to the evaporator and configured to sense the amount of the liquid sample in the gas to measure the concentration of the volatile or gaseous compound; vaporizing or evaporating the liquid to form a compound-rich gas; moving the compound-rich gas to the one or more gas sensors; collecting gas sensor readings; integrating the collected gas sensor readings; normalizing integration of the integrated gas sensor readings; computing output of the concentration of a volatile or gaseous compound in the liquid based on calibration.

One or more implementations of the aspect of the present disclosure described immediately above includes one or more of the following: vaporizing or evaporating the liquid to form a compound-rich gas includes vaporizing or evaporating the liquid with an ultrasonic disk to form a compound-rich gas; metering one or more of the liquid and the gas via one or more of volumetric measurement of the liquid, limiting evaporation or vaporization time, volumetric airflow measurement, and mass measurement; computing output includes computing output of the concentration of a volatile or gaseous compound in the liquid based on calibration independent of atmospheric temperature, atmospheric pressure, sample temperature, sample viscosity, off-target compounds, or other environmental variables in the liquid; computing output includes computing output of the concentration of a volatile or gaseous compound in the liquid negligibly unaffected by viscosity, carbonation, turbidity, sugar content, or acid content of the liquid; the liquid is a beverage including alcohol and vaporizing or evaporating the liquid includes vaporizing or evaporating the beverage including alcohol to form a compound-rich gas, and computing output includes computing output of an alcohol level in the liquid based on calibration; and/or the liquid is kombucha and vaporizing or evaporating the liquid includes vaporizing or evaporating the kombucha to form a compound-rich gas, and computing output includes computing output of an alcohol level in the liquid based on calibration.

### BRIEF DESCRIPTION OF THE DRAWINGS

In order to describe the manner in which the present disclosure can be implemented, a more particular description will be rendered by reference to specific implementations thereof which are illustrated in the appended drawings. For better understanding, the like elements have been designated by like reference numbers throughout the various accompanying figures. Understanding that these drawings depict only typical implementations of the invention and are not therefore to be considered to be limiting of its scope, the invention will be described and explained with additional specificity and detail through the use of the accompanying drawings in which:

FIG. 1 is a functional block diagram of a detection system designed to find the alcohol content of a liquid sample in accordance with an embodiment of the disclosure;

FIG. 2 is a perspective view of a detection system designed to find the alcohol content of a liquid sample in accordance with another embodiment of the disclosure;

FIG. 3 is a perspective view of an embodiment of an ultrasonic disk of the detection system of FIG. 2 shown evaporating a liquid sample;

FIG. 4 is a perspective view of an embodiment of a glass vial with a nichrome wire coil that can be used to evaporate a liquid sample;

FIG. 5 is a perspective view of embodiments of gas sensors that may be used to identify volatile compounds like ethanol;

FIG. 6 is a flowchart of an exemplary method of determining volatile compounds by metered sample evaporation.

FIG. 7 is a block diagram illustrating an example wired or wireless processor enabled device that may be used in connection with various embodiments described herein.

### DETAILED DESCRIPTION

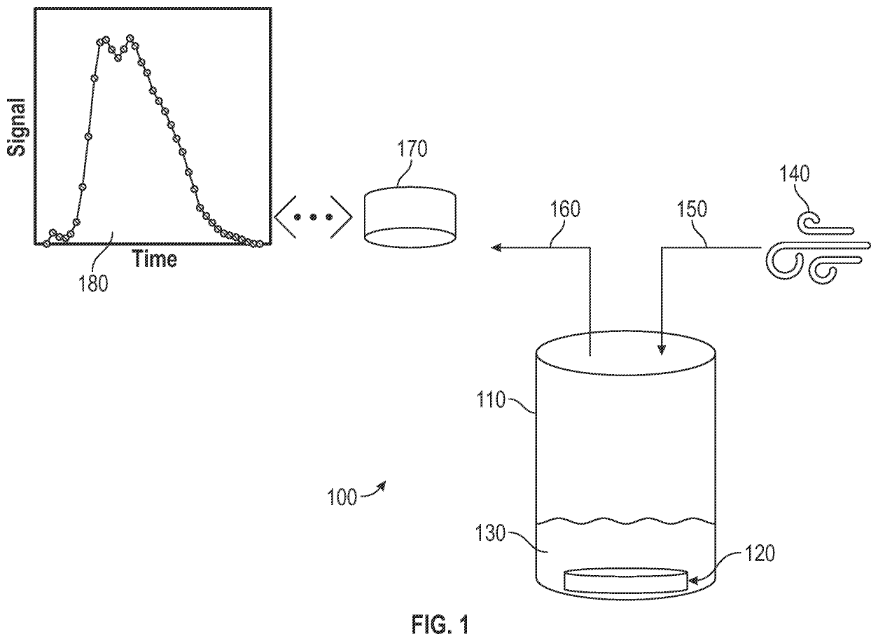

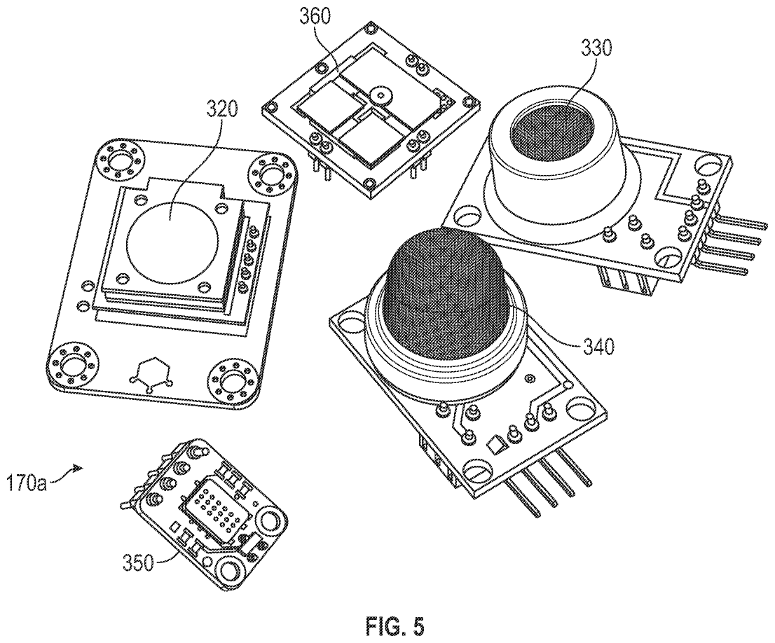

In this disclosure, as illustrated in FIGS. 1-6, specific terminology is employed for the sake of clarity. The invention as claimed in this application, however, is not intended to be limited to specific terminology so selected, and it is to be understood that each specific element includes all technical equivalents that operate in a similar manner to accomplish similar functions.

With reference to FIG. 1, an embodiment of a detection system 100 designed to find the alcohol content of a liquid sample will be described. The detection system 100 includes sample fluid container/vial/reservoir 110 including an evaporator 120 that vaporizes/evaporates a liquid sample (e.g., liquid sample including alcohol) 130, a fan/air pump 140, an inlet line 150, an outlet line 160, and gas sensor(s) 170. The detection system 100 measures vaporizable compounds from the liquid sample 130 and calculates total compounds using integration of sensor data. To find the concentration of a vaporizable compound in the liquid sample 130, a measured amount of liquid sample 130 or gas is carried over the gas sensor(s) 170. In some cases, the liquid sample 130 may contain an amount of analyte that would saturate the gas sensor(s) 170. In these cases and others, one or more embodiments of the detection system 100 may include air ducts that specifically dilute or change the speed of the gas. The integral or sum of measurements during a period of time is calculated and shown at calculation/graph 180. This calculation/graph 180 is used to determine properties of the liquid sample 130. If the liquid sample 130 is being evaporated or vaporized, the fan/air pump 140 blow fresh gas into the container/vial 110 through inlet 150. Gas passes out of the container/vial 110 via the outlet line 160 and over the gas sensor(s) 170. The detection system 100 and described method provide readings independent of sample temperature, atmospheric pressure, atmospheric temperature, sample viscosity, and off-target compounds in the liquid sample 130.

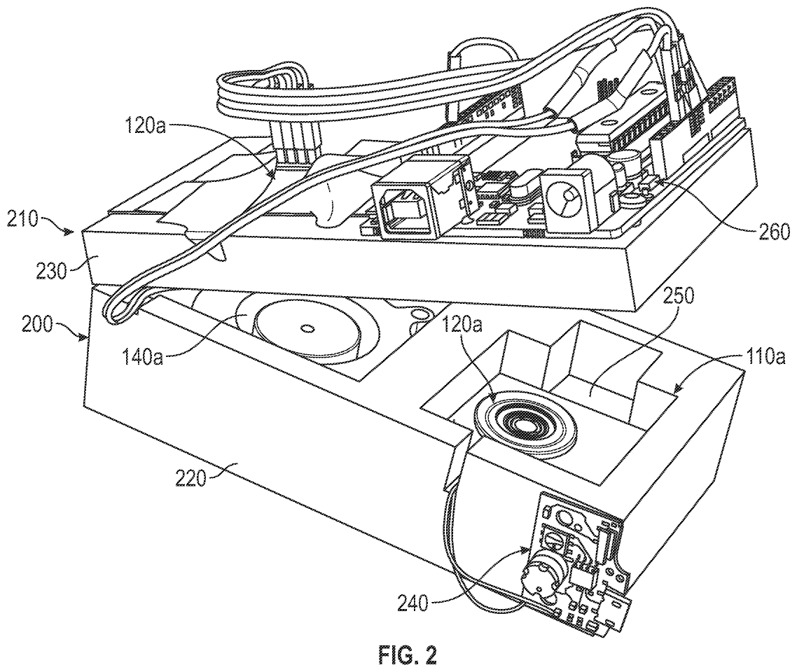

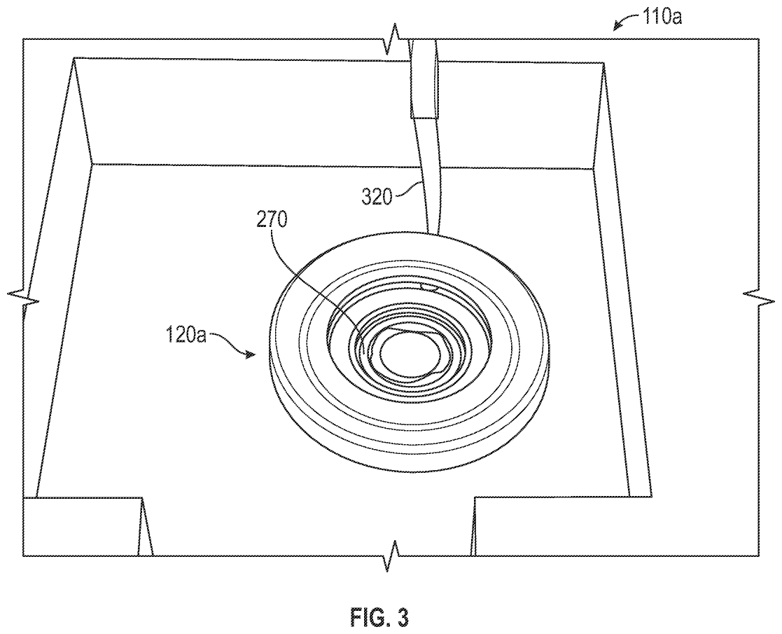

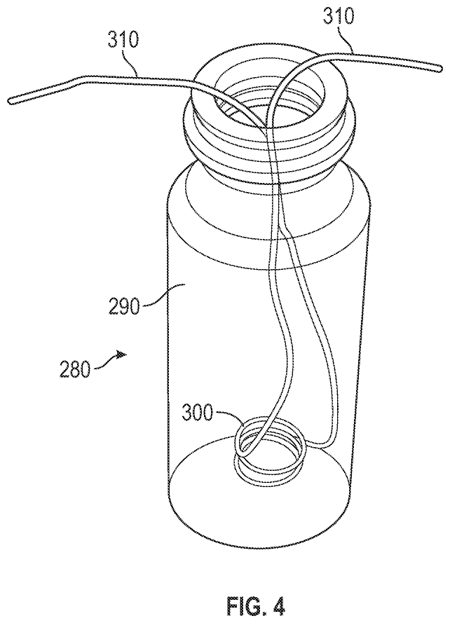

With reference to FIG. 2, another embodiment of a detection system 200 designed to find the alcohol content of a liquid sample will be described. Like elements to those shown and described with respect to detection system 100 and FIG. 1 will be shown with the same reference numbers, but with an “a” suffix and the above description is incorporated herein. The detection system 200 includes a housing 210 with a base 220 and a top 230. The base 220 includes a fluid reservoir 110a including an evaporator 120a with connected driver circuit 240, a fan 140a, and a gas line/air ducting 250. The top 230 includes a gas sensor(s) 170a and a microcontroller 260. The evaporator 120a (FIG. 3) includes a replaceable ultrasonic disk 270. In an alternative embodiment, as shown in FIG. 4, evaporation can occur with an evaporator 280 including a small vial 290, which allows a small sample to be easily contained, a nichrome wire 300 including wire connectors 310, one of which is connected to a power source, resulting in a hot wire that can be in direct or indirect contact with the liquid sample. Evaporation may also be controlled with a temperature probe and PID control. Other vaporization or evaporation methods may be applicable.

An 100 μL alcohol sample is completely vaporized with the ultrasonic disk 270 driven by the driver circuit 240 into a jet of mist/vapor 320 (FIG. 3) in 30 seconds. The fan 140a blows fresh air through gas line/air ducting 250 to carry the alcohol-rich air through further ducting to be directed over the gas sensor (e.g., MiCS5524 gas sensor) 170a. Signal from the sensor 170a is collected every second and is processed by the microcontroller (e.g., Arduino Uno) 260. Data processing may occur externally on a laptop that receives data from the microcontroller 260. Calculations are performed by taking the sum of all datapoints received during the sample measurement period. This sum is consistent independent of sample temperature, atmospheric temperature, atmospheric pressure, or any other environmental variable. By measuring known alcohol standards to generate a calibration curve, any unknown alcohol sample of any alcohol level can be calculated. These readings are not affected by common sample issues including, but not limited to viscosity, carbonation, turbidity, sugar content, or acid content.

An essential aspect of the detection systems 100, 200 is metered vaporization or evaporation. This is typically achieved by volumetric measurement of the sample, but could also be achieved by limiting sample evaporation/vaporization time, volumetric airflow measurement, or mass measurement. Vaporization is preferably performed by the ultrasonic disk 270 that results in rapid vaporization yielding the jet of mist/vapor 320 (FIG. 3) or pulsed, slower vaporization.

Another essential component of the detection systems 100, 200 is/are gas sensor(s) 170, 170a. Gas sensors 170, 170a can be sensitive to carbon dioxide, ethanol, carbon monoxide, nitrogen dioxide, ammonia, hydrogen sulfide, various volatile organic compounds, and many other gases/chemicals. No gas sensor is perfectly specific and thus all gas sensors are sensitive to off-target compounds. The detection systems 100, 200 include, but are not limited to, one or more of MQ-7 sensor 330, MQ-4 sensor 340, MiCS5524 gas sensor 350, alcohol 2 click ethanol gas sensor 360, and gravity alcohol sensor 370. These sensors 170, 170a may be swapped by the user to change the detection range of the detection systems 100, 200. In alternative embodiments, other types of gas sensor(s) 170, 170a are used.

In another embodiment of the detection systems 100, 200 removable sensor cartridges carrying the gas sensor(s) 170, 170a are used to expand the detection range and chemicals that can be detected. This allows the user to easily replace old sensors, detect different compounds, and expand the detection range of the detection systems 100, 200. The geometry of the gas ducting lines 150, 160, 250 may also be adjusted to dilute airflow, change air flowrate, or change the temperature of the gas stream. Multiple sensors can also be utilized for precise measurements. Various carrier gasses can be used like air, nitrogen, hydrogen, helium, and others. Single or combinations of gas sensors 170, 170a may be used in the detection systems 100, 200 with finite models or machine learning methods. This would allow for detection of liquid properties beyond single molecules. In alternative embodiments, the liquid sample 130 could be blood, urine, or another bodily fluid and a sensor or sensor array with data processing methods could be used to detect disease.

Standardized liquid extraction procedures of solid material like plant matter, food, or tissue can be used to prepare liquid samples 130 for analysis to detect viral, fungal, or bacterial contamination, production of metabolites, phenotypes of interest, or other properties.

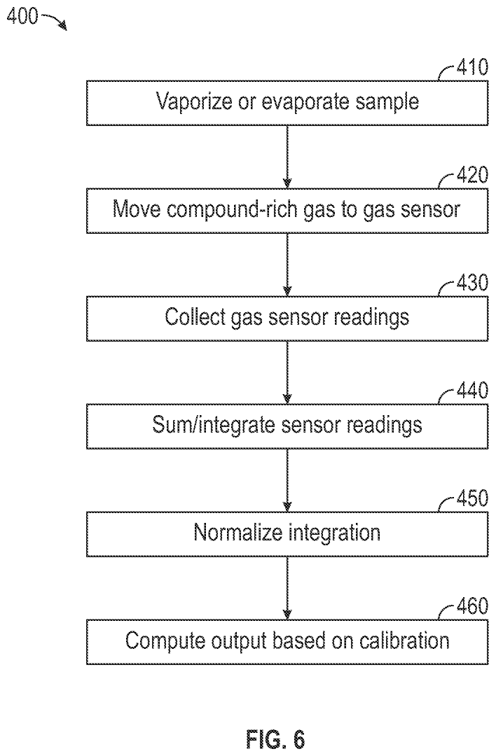

With reference to FIG. 6, a method 400 of using the detection systems 100, 200 to measure vaporizable compounds from the liquid sample 130 comprises, at step 410, vaporizing or evaporating the liquid sample 130, at step 420, moving a compound-rich gas to the gas sensor(s) 170, 170a, at step 430, collecting gas sensor readings, at step 440, summing/integrating sensor readings, at step 450, normalizing integration, and, at step 450, computing output based on calibration. In a preferred embodiment, the duration of the method 500 is less than five minutes. In an alternative preferred embodiment, the duration of the method 500 is one minute or less.

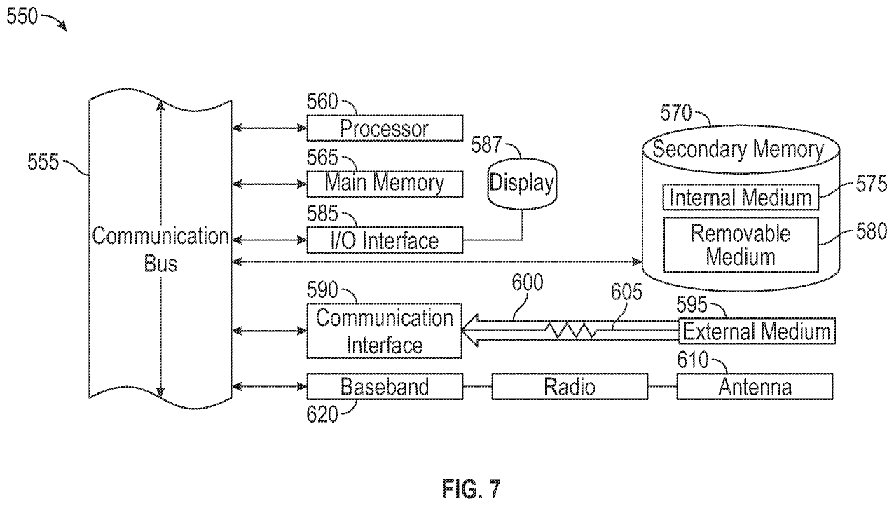

FIG. 7 is a block diagram illustrating an example wired or wireless system 550 that may be used in connection with various embodiments described herein. For example, the system 550 may be used as or in conjunction with the driver circuit 240, the microcontroller 260, and/or any other controller/control function/method described herein with respect to the detection systems 100, 200. The system 550 can be a conventional personal computer, computer server, personal digital assistant, smart phone, tablet computer, or any other processor enabled device that is capable of wired or wireless data communication. Other computer systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

System 550 preferably includes a main memory 565 and may also include a secondary memory 570. The main memory 565 provides storage of instructions and data for programs executing on the processor 560. The main memory 565 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 570 may optionally include an internal memory 575 and/or a removable medium 580, for example a floppy disk drive, a magnetic tape drive, a compact disc (“CD”) drive, a digital versatile disc (“DVD”) drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 570. Examples of external storage medium 595 may include an external hard disk drive or an external optical drive, or and external magneto-optical drive.

Other examples of secondary memory 570 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage media 580 and communication interface 590, which allow software and data to be transferred from an external medium 595 to the system 550.

System 550 may also include an input/output (“I/O”) interface 585. The I/O interface 585 facilitates input from and output to external devices. For example the I/O interface 585 may receive input from a keyboard or mouse and may provide output to a display 587. The I/O interface 585 is capable of facilitating input from and output to various alternative types of human interface and machine interface devices alike.

System 550 may also include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a modem, a network interface card (“NIC”), a wireless data card, a communications port, a PCMCIA slot and card, an infrared interface, and an IEEE 1394 fire-wire, just to name a few.

Communication interface 590 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present disclosure as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

The system 550 also includes optional wireless communication components that facilitate wireless communication over a voice and over a data network. The wireless communication components comprise an antenna system 610, a radio system 615 and a baseband system 620. In the system 550, radio frequency (“RF”) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present disclosure as previously described. For example, data storage areas 565 may include various software modules (not shown) that are executable by processor 560.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The above figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

I claim:

1. A detection system for measuring the concentration of a volatile or gaseous compound in a liquid, comprising:
a reservoir configured to receive the liquid;
an evaporator configured to evaporate or vaporize the liquid received in the reservoir into a gas including an amount of a liquid sample;
one or more gas sensors coupled to the evaporator and configured to sense the amount of the liquid sample in the gas to measure the concentration of the volatile or gaseous compound.

2. The detection system of claim 1, wherein the one or more sensors are configured to provide a varying voltage output proportional to a compound in the amount of the liquid sample in the gas near the one or more sensors, the detection system further including a controller configured to receive sensor values from the one or more gas sensors and calculate an amount of compound in the liquid based on an integral of the sensor values received from the one or more gas sensors.

3. The detection system of claim 2, wherein the evaporator includes an ultrasonic disk configured to vaporize the liquid.

4. The detection system of claim 3, wherein the ultrasonic disk is configured to vaporize the liquid into one of faster vaporization yielding a jet of vapor mist and slower, pulsed vaporization.

5. The detection system of claim 1, wherein the detection system is configured to measure the concentration of the volatile or gaseous compound in the liquid independent of atmospheric temperature, atmospheric pressure, sample temperature, sample viscosity, off-target compounds, or other environmental variables in the liquid.

6. The detection system of claim 1, wherein the detection system is configured to measure the concentration of the volatile or gaseous compound in the liquid negligibly unaffected by viscosity, carbonation, turbidity, sugar content, or acid content of the liquid.

7. The detection system of claim 1, wherein the detection system is configured to meter one or more of the liquid and the gas via one or more of volumetric measurement of the liquid, limiting evaporation or vaporization time, volumetric airflow measurement, and mass measurement.

8. A method of measuring the concentration of a volatile or gaseous compound in a liquid, comprising:
providing the detection system of claim 1;
vaporizing or evaporating the liquid to form a compound-rich gas;
moving the compound-rich gas to the one or more gas sensors;
collecting gas sensor readings;
integrating the collected gas sensor readings;
normalizing integration of the integrated gas sensor readings;
computing output of the concentration of a volatile or gaseous compound in the liquid based on calibration.

9. The method of claim 8, wherein vaporizing or evaporating the liquid to form a compound-rich gas includes vaporizing or evaporating the liquid with an ultrasonic disk to form a compound-rich gas.

10. The method of claim 8, further comprising metering one or more of the liquid and the gas via one or more of volumetric measurement of the liquid, limiting evaporation or vaporization time, volumetric airflow measurement, and mass measurement.

11. The method of claim 8, wherein computing output includes computing output of the concentration of a volatile or gaseous compound in the liquid based on calibration independent of atmospheric temperature, atmospheric pressure, sample temperature, sample viscosity, off-target compounds, or other environmental variables in the liquid.

12. The method of claim 8, wherein computing output includes computing output of the concentration of a volatile or gaseous compound in the liquid negligibly unaffected by viscosity, carbonation, turbidity, sugar content, or acid content of the liquid.

13. The method of claim 8, wherein the liquid is a beverage including alcohol and vaporizing or evaporating the liquid includes vaporizing or evaporating the beverage including alcohol to form a compound-rich gas, and computing output includes computing output of an alcohol level in the liquid based on calibration.

14. The method of claim 13, wherein the liquid is kombucha and vaporizing or evaporating the liquid includes vaporizing or evaporating the kombucha to form a compound-rich gas, and computing output includes computing output of an alcohol level in the liquid based on calibration.
